(19) United States
(12) Patent Application Publication
(10) Pub. No.: US 2015/0355185 A1
(43) Pub. Date: Dec. 10, 2015

(54) USING GALECTIN-BINDING CARBOHYDRATES AS PREDICTORS OF MELANOMA PROGRESSION AND METASTASIS

(71) Applicant: The Brigham and Women's Hospital, Inc., Boston, MA (US)

(72) Inventor: Charles J. DIMITROFF, Williamsville, NY (US)

(73) Assignee: THE BRIGHAM AND WOMEN'S HOSPITAL, INC., Boston, MA (US)

(21) Appl. No.: 14/760,558

(22) PCT Filed: Jan. 16, 2014
(86) PCT No.: PCT/US2014/011764

§ 371 Date: Jul. 13, 2015

Related U.S. Application Data
(60) Provisional application No. 61/753,228, filed on Jan. 16, 2013.
(60) Provisional application No. 61/753,242, filed on Jan. 16, 2013.

(51) Int. Cl. G01N 33/574

(52) U.S. Cl. CPC G01N 33/57492; G01N 33/5743; G01N 33/57449; G01N 2333/705; G01N 2496/00

(57) ABSTRACT

Disclosed herein are assays and methods to determine tumor malignancy, for example, in melanoma or ovarian carcinoma, by determining the expression level of Gal-1 ligands. Also provided are methods to assess the metastatic potential of a tumor.

### CROSS-REFERENCE TO RELATED APPLICATION

This application claims benefit under 35 U.S.C. §119(e) of U.S. Provisional Application No. 61/753,228 filed Jan. 16, 2013 and U.S. Provisional Application No. 61/753,242 filed Jan. 16, 2013, the contents of which are incorporated herein by reference in its entirety.

### GOVERNMENT SUPPORT

This invention was made with government support under grant R01 CA173610 awarded by the National Institutes of Health/National Cancer Institute. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Jan. 15, 2014, is named 043214-076642-PCT SL.txt and is 20,247 bytes in size.

### TECHNICAL FIELD

The present disclosure relates generally to biomarkers for cancer diagnostics and treatment.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Since metastatic melanoma often results in a deadly outcome, determining whether a dysplastic nevi is malignant or will progress to a malignancy and whether a primary melanoma will metastasize are fundamental to diagnosing melanomas and prognosticating clinical outcome.

One of the challenges for a dermatopathologist is determining which dysplastic nevi, if any, are likely to progress to malignancy and whether malignant melanoma will progress to metastasis. Current immunohistological markers of melanoma cells, including MART-1, MITF and S100, are expressed on all melanocytic lineages, and thus tell us little with regard to biological potential. Though analyses of other histologic features, such as tumor depth, presence and number of mitoses, and presence or absence of ulceration, are valuable (Balch, C. M., et al., J Clin Oncol, 2001, 19, 3635-48), discerning aggressive from non-aggressive melanomas is still difficult.

Accordingly, a great need exists in the art for new methods to discern aggressive melanomas from non-aggressive melanomas, and new methods that permit a physician to assess or predict the metastatic potential of melanomas.

### SUMMARY

The following embodiments and aspects thereof are described and illustrated in conjunction with systems, compositions and methods which are meant to be exemplary and illustrative, not limiting in scope.

The invention is based, in part, on the following surprising discoveries: first, melanoma cells express galectin-1 (Gal-1) ligands on the cell surface; and second, expression levels of Gal-1 ligands are tumor-stage dependent, meaning that Gal-1 ligands are overexpressed on malignant melanoma cell surface but not on normal melanocytes or melanocytes in compound nevi. By establishing the role of Gal-1 ligands in tumor malignancy development, assays and methods are described herein to exploit Gal-1 ligands for cancer diagnosis and treatment. In particular, the assays and methods provide needed solutions to discern aggressive melanomas from non-aggressive ones.

One aspect of the invention relates to an assay comprising measuring, in a sample obtained from a subject, an expression level of Gal-1 ligands, and determining that the subject has cancer if the expression level of Gal-1 ligands is higher than a reference level.

In some embodiments, the expression level of Gal-1 ligands is measured by immunofluorescence, histology, or immunohistochemistry.

In some embodiments, the expression level of Gal-1 ligands is measured by contacting the sample with a Gal-1-human Fc (Gal-1hFc) fusion protein.

In some embodiments, the tumor expresses Gal-1 ligands.

In some embodiments, the tumor is a skin tumor, and the cancer is malignant melanoma.

In some embodiments, the tumor is an ovarian tumor, and the cancer is ovarian carcinoma.

In some embodiments, the sample is obtained by performing a biopsy on a subject.

In some embodiments, the subject is a mammal.

In some embodiments, the mammal is a human.

A related aspect of the invention regards methods that use the assays described herein to determine the malignancy of a tumor. The method comprises assaying a sample obtained from a subject for an expression level of Gal-1 ligands, and determining that the subject has cancer if the expression level of Gal-1 ligands is higher than a reference level.

Another related aspect of the invention regards a method of assessing or predicting a metastatic potential of a tumor from a subject, comprising the following steps: first, measuring, at a first time point, a first expression level of Gal-1 ligands of a first sample from the subject; second, measuring, at a second time point, a second expression level of Gal-1 ligands of a second sample from the subject, wherein the second time point is later than the first time point; third, comparing the first expression level with the second expression level, and if the second expression level is higher than the first expression level, the tumor is likely to metastasize.

In some embodiments, the expression level of Gal-1 ligands is measured by an assay comprising contacting the sample with Gal-1hFc, washing the sample to remove excess unbound Gal-1hFc, and detecting a presence or intensity of a detectable signal.

Yet another aspect of the invention relates to cancer treatment by targeting Gal-1 ligands of cancer cells. The method comprises determining whether a subject has a cancer that expresses Gal-1 ligands by using the assays or methods described herein, and administering to the subject a composition that targets Gal-1 ligands.

In some embodiments, the composition is toxic to cancer cells expressing Gal-1 ligands.

A further aspect of the invention relates to a composition that comprises a tumor cell and Gal-1hFc, and Gal-1hFc is bound to the tumor cell. In some embodiments, Gal-1hFc is bound to the tumor cell through the Gal-1 ligands expressed on the tumor cell surface.

Relatedly, a method of making a composition comprising a tumor cell bound to Gal-1hFc, comprises contacting the tumor cell with Gal-1hFc, and washing the tumor cell to remove excess unbound Gal-1hFc.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 depicts, in accordance with various embodiments of the present invention, a graphical presentation showing hypothetical consequences following Galectin-1 (Gal-1)-binding to melanoma cells. Upon Gal-1 binding in an autocrine-like setting, Gal-1 may impart patho-biological activities, such as invasion, adhesion, immune evasion and angiogenesis, which can contribute to the malignant potential of melanoma cells.

FIG. 2 depicts, in accordance with various embodiments of the present invention, dual immunofluorescence of Galectin-1 (Gal-1) ligands and S100 on normal human skin and on benign or malignant melanocytic tumors. Gal-1 ligands and S100 were stained with Gal-1hFc and anti-S100 Ab on formalin fixed paraffin-embedded sections of normal, human skin or on cutaneous benign (dysplastic nevi) or malignant melanocytic tumors. Combined dual staining was noted and incremental on dermal nests of a severely-dysplastic nevus, on superficial and vertical growth variants of cutaneous malignant melanoma, and on distant metastases. Sections were counterstained with nuclear stain DAPI.

FIGS. 3A-3F depicts, in accordance with various embodiments of the present invention, that Galectin-1-Human Fc (Gal-1hFc) chimera can be a powerful new tool for detecting Galectin-1 (Gal-1) ligands. To create a structural homodimer mimetic of native Gal-1 (FIG. 3A), we genetically fused full-length mouse Gal-1 to human Fc in frame (FIG. 3B) and to structurally encourage a homodimeric structure that is optimal for Gal-1 ligand binding and induction of Gal-1 ligand-mediated cellular activities. A non-Gal-1 ligand-binding mutant dmGal-1hFc (FIG. 3C) that contains two mutations in key AA residues in the carbohydrate-recognition domain (CRD). These chimeras are effectively used in bioassays, such as flow cytometry (FIG. 3D), Western blotting (FIG. 3E) and immunofluorescence (FIG. 3F) to bind/detect Gal-1 ligands.

FIGS. 4A-4B depicts, in accordance with various embodiments of the present invention, dual immunofluorescence of Galectin-1 (Gal-1) ligands and CD3 (FIG. 4A) or S100 (FIG. 4B) on metastatic melanoma. Gal-1 ligands and CD3 or S100 were stained with Gal-1hFc and anti-CD3 Ab or anti-S100 Ab on formalin fixed paraffin-embedded sections of human metastatic melanoma. Dual staining was noted in both Gal-1hFc/CD3 and Gal-1hFc/S100 combinations. In particular, there was remarkable Gal-1hFc/S100 dual staining illustrated on metastatic melanoma cells, indicating high expression of Gal-1 ligands. Sections were counterstained with nuclear stain DAPI.

FIGS. 5A-5D depicts, in accordance with various embodiments of the present invention, immunofluorescence and analysis of Galectin-1 (Gal-1) ligands on benign and primary malignant and metastatic melanocytic tumors. Gal-1 ligands were stained with Gal-1hFc on formalin fixed paraffin-embedded sections of human metastatic melanoma (FIG. 5A), primary melanomas (FIG. 5B) or benign nevi (FIG. 5C). Sections were counterstained with nuclear stain DAPI. FIG. 5D shows fluorescence analysis performed using Spot Advanced software, and representative core fields at 10× magnification (encompassing >85% of each core) were analyzed using semi-quantitative intensity analysis with NIH Image J software.

FIGS. 6A-6C depicts, in accordance with various embodiments of the present invention, dual immunofluorescence of Galectin-1 (Gal-1) ligands and S100 on normal human skin and malignant melanocytic tumors. Gal-1 ligands and S100 were stained with Gal-1hFc and anti-S100 Ab on formalin fixed paraffin-embedded sections of normal human skin (FIG. 6A) or on cutaneous radial growth phase (RGP) (FIG. 6B) or vertical growth phase (VGP) (FIG. 6C) malignant melanocytic tumors. Combined dual staining was noted on superficial and vertical growth variants of cutaneous malignant melanoma. There was no staining with control non-binding dmGal-1hFc or with Gal-1hFc on normal skin. Sections were counterstained with nuclear stain DAPI and images taken at 10× magnification.

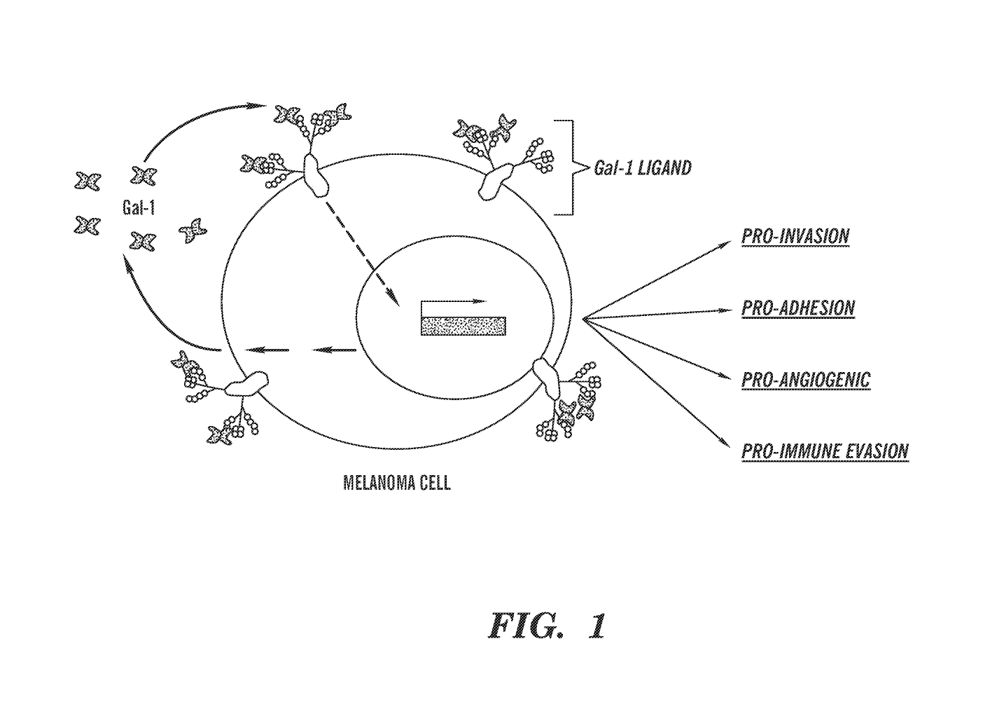

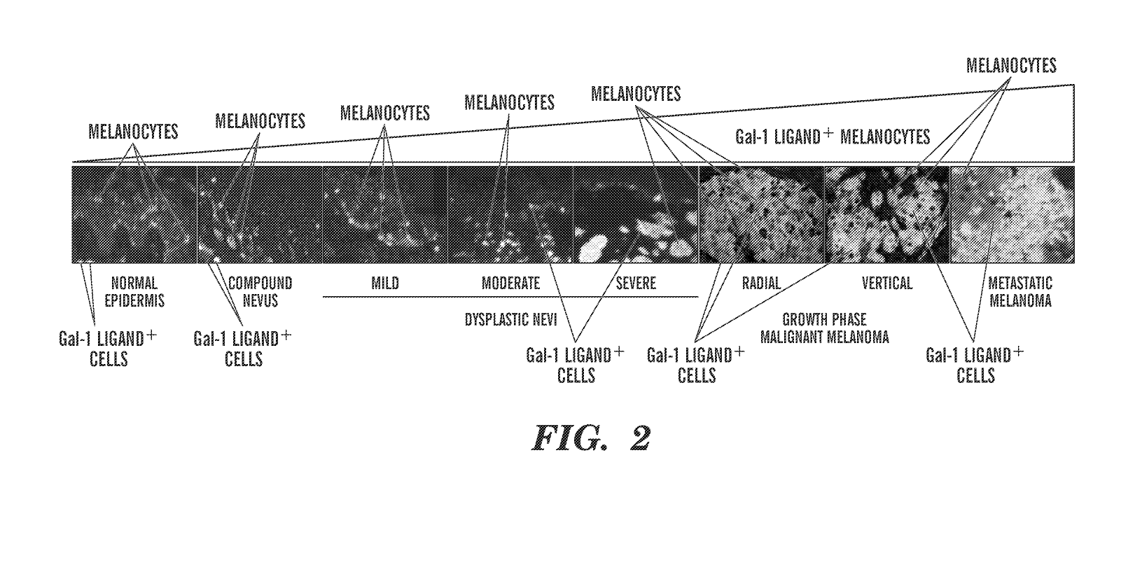

### DETAILED DESCRIPTION

All references cited herein, including the references cited therein, are incorporated by reference in their entirety as though fully set forth. Unless defined otherwise, technical and scientific terms used herein have the same meaning as commonly understood by one of ordinary skill in the art to which this invention belongs. The practice of the present invention will employ, unless indicated specifically to the contrary, conventional methods of molecular biology and recombinant DNA techniques within the skill of the art, many of which are described below for the purpose of illustration. Such techniques are fully explained in the literature. See, e.g., Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001), Sambrook, et al., Molecular Cloning: A Laboratory Manual (3rd Edition, 2000); Sambrook and Russel, Molecular Cloning: A Laboratory Manual 3rd ed., Cold Spring Harbor Laboratory Press (Cold Spring Harbor, N.Y. 2001), DNA Cloning: A Practical Approach, vol. I & II (D. Glover, ed.); Oligonucleotide Synthesis (N. Gait, ed., 1984); Oligonucleotide Synthesis: Methods and Applications (P. Herdewijn, ed., 2004); Nucleic Acid Hybridization (B. Hames & S. Higgins, eds., 1985); Nucleic Acid Hybridization: Modern Applications (Buzdin and Lukyanov, eds., 2009); Transcription and Translation (B. Hames & S. Higgins, eds., 1984); Animal Cell Culture (R. Freshney, ed., 1986); Freshney, R. I. (2005) Culture of Animal Cells, a Manual of Basic Technique, 5th Ed. Hoboken N.J., John Wiley & Sons; B. Perbal, A Practical Guide to Molecular Cloning (3rd Edition 2010); Farrell, R., RNA Methodologies: A Laboratory Guide for Isolation and Characterization (3rd Edition 2005), Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-3, 4-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, (2002, Cold Spring Harbor Laboratory, ISBN 0-87969-630-3) provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein the term “comprising” or “comprises” is used in reference to compositions, methods, and respective component(s) thereof, that are useful to an embodiment, yet open to the inclusion of unspecified elements, whether useful or not.

The singular terms “a,” “an,” and “the” include plural referents unless context clearly indicates otherwise. Similarly, the word “or” is intended to include “and” unless the context clearly indicates otherwise. The abbreviation, “e.g.” is derived from the Latin exempli gratia, and is used herein to indicate a non-limiting example. Thus, the abbreviation “e.g.” is synonymous with the term “for example.”

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of cancer progression, delay or slowing of metastasis or invasiveness, and amelioration or palliation of symptoms associated with the cancer. Treatment also includes a decrease in mortality or an increase in the lifespan of a subject as compared to one not receiving the treatment.

As used herein, the term “administering,” refers to the placement an agent as disclosed herein into a subject by a method or route which results in at least partial localization of the agents at a desired site.

The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene (e.g. mRNA), and polypeptides obtained by translation of mRNA transcribed from a gene.

A “cancer” or “tumor” as used herein refers to an uncontrolled growth of cells which interferes with the normal functioning of the bodily organs and systems. A subject that has a cancer or a tumor is a subject having objectively measurable cancer cells present in the subject's body. Included in this definition are benign and malignant cancers, as well as dormant tumors or micrometastatses. Cancers which migrate from their original location and seed vital organs can eventually lead to the death of the subject through the functional deterioration of the affected organs. As used herein, the term “carcinoma” refers to a cancer arising from epithelial cells. As used herein, the term “invasive” refers to the ability to infiltrate and destroy surrounding tissue. Melanoma is an invasive form of skin tumor.

The term “sample” or “biological sample” as used herein denotes a sample taken or isolated from a biological organism, e.g., a tumor sample from a subject. Exemplary biological samples include, but are not limited to, a biofluid sample; serum; plasma; urine; saliva; a tumor sample; a tumor biopsy and/or tissue sample etc. The term also includes a mixture of the above-mentioned samples. The term “sample” also includes untreated or pretreated (or pre-processed) biological samples. In some embodiments, a sample can comprise one or more cells from the subject. In some embodiments, a sample can be a tumor cell sample, e.g. the sample can comprise cancerous cells, cells from a tumor, and/or a tumor biopsy.

The term “functional” when used in conjunction with “derivative” or “variant” or “fragment” refers to a polypeptide which possess a biological activity that is substantially similar to a biological activity of the entity or molecule of which it is a derivative or variant or fragment thereof. By “substantially similar” in this context is meant that at least 25%, at least 35%, at least 50% of the relevant or desired biological activity of a corresponding Gal-1hFc fusion protein is retained.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein.

Preferably, the subject is a mammal. The mammal can be a human, non-human primate, mouse, rat, dog, cat, horse, or cow, but are not limited to these examples. In addition, the methods described herein can be used to treat domesticated animals and/or pets.

A subject can be one who has been previously diagnosed with or identified as suffering from or having a condition in need of monitoring (e.g., skin tumor) or one or more complications related to such a condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition or one or more complications related to a condition or a subject who does not exhibit risk factors. A “subject in need” of treatment for a particular condition can be a subject having that condition, diagnosed as having that condition, or at risk of developing that condition.

As used herein, the word “toxic” means effects on cells (e.g., tumor cells) that result in cell death, reduced ability to divide, or cell population reduction.

As used herein, the term “antibody” refers to an intact immunoglobulin or to a monoclonal or polyclonal antigen-binding fragment with the Fc (crystallizable fragment) region or FcRn binding fragment of the Fc region, referred to herein as the “Fc fragment” or “Fc domain”. Antigen-binding fragments may be produced by recombinant DNA techniques or by enzymatic or chemical cleavage of intact antibodies. Antigen-binding fragments include, inter alia, Fab, Fab′, F(ab′)2, Fv, dAb, and complementarity determining region (CDR) fragments, single-chain antibodies (scFv), single domain antibodies, chimeric antibodies, diabodies, tetrabodies and other multimerized scFv moieties and polypeptides that contain at least a portion of an immunoglobulin that is sufficient to confer specific antigen binding to the polypeptide. The Fc domain includes portions of two heavy chains contributing to two or three classes of the antibody. The Fc domain may be produced by recombinant DNA techniques or by enzymatic (e.g. papain) cleavage or via chemical cleavage of intact antibodies.

The term “antibody fragment,” as used herein, refer to a protein fragment that comprises only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 341, 544-546 (1989)) which consists of a VH domain; (vii) isolated CDR regions; (viii) F(ab′)2 fragments, a bivalent fragment including two Fab′ fragments linked by a disulphide bridge at the hinge region; (ix) single chain antibody molecules (e.g., single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (VH-CH1-VH-CH1) which, together with complementary light chain polypeptides, form a pair of antigen binding regions (Zapata et al. Protein Eng. 8(10):1057-1062 (1995); and U.S. Pat. No. 5,641,870).

As described herein, an “antigen” is a molecule that is bound by a binding site on a polypeptide agent, such as an antibody or antibody fragment thereof. Typically, antigens are bound by antibody ligands and are capable of raising an antibody response in vivo. An antigen can be a polypeptide, protein, nucleic acid, lipid or other molecule. In the case of conventional antibodies and fragments thereof, the antibody binding site as defined by the variable loops (L1, L2, L3 and H1, H2, H3) is capable of binding to the antigen. The term “antigenic determinant” refers to an epitope on the antigen recognized by an antigen-binding molecule, and more particularly, by the antigen-binding site of said molecule.

An “Fv” fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) has the ability to recognize and bind antigen, although usually at a lower affinity than the entire binding site.

As used herein, an “epitope” can be formed both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An “epitope” includes the unit of structure conventionally bound by an immunoglobulin VH/VL pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein. In various embodiments, an epitope may be protein, peptide, nucleic acid, lipid, other molecules or combinations thereof.

The term “monoclonal antibody” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method. For example, the monoclonal antibodies to be used in accordance with the invention can be made by the hybridoma method first described by Kohler et al., Nature 256:495 (1975), or can be made by recombinant DNA methods (see, e.g., U.S. Pat. No. 4,816,567). The “monoclonal antibodies” can also be isolated from phage antibody libraries using the techniques described in Clackson et al., Nature 352:624-628 (1991) or Marks et al., J. Mol. Biol. 222:581-597 (1991), for example.

As used herein, “stored” refers to a process for encoding information on the storage module. Those skilled in the art can readily adopt any of the presently known methods for recording information on known media to generate manufactures comprising expression level information.

The term “statistically significant” or “significantly” refers to statistical significance and generally means a two standard deviation (2 SD) below normal, or lower, concentration of the marker. The term refers to statistical evidence that there is a difference. It is defined as the probability of making a decision to reject the null hypothesis when the null hypothesis is actually true. The decision is often made using the p-value.

The term “computer” can refer to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” may refer to any storage device used for storing data accessible by a computer, as well as any other means for providing access to data by a computer. Examples of a storage-device-type computer-readable medium include: a magnetic hard disk; a floppy disk; an optical disk, such as a CD-ROM and a DVD; a magnetic tape; a memory chip.

The term “software” is used interchangeably herein with “program” and refers to prescribed rules to operate a computer. Examples of software include: software; code segments; instructions; computer programs; and programmed logic.

The term a “computer system” may refer to a system having a computer, where the computer comprises a computer-readable medium embodying software to operate the computer.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

The invention is based, in part, on the surprising discovery that Gal-1 ligands are overexpressed on malignant melanoma cell surface but not on normal melanocytes or melanocytes in compound nevi. This discovery offers as yet unavailable methods to discern aggressive melanomas from non-aggressive ones, which are highly valuable in clinical applications. As it is estimated that one in five Americans will develop skin cancer in the course of a lifetime, the assays and methods described herein can potentially benefit millions of lives. More broadly, the invention can be applicable in any cancer that expresses Gal-1 ligands.

It is known that tumor cells display aberrant levels of cell surface glycans that often facilitate malignant behavior. Functional expression of these glycans is linked to a number of behaviors associated with malignant progression, such as adhesion, invasion, angiogenesis and metastasis. Galectin-1 (Gal-1), which is abundantly produced by melanoma cells, facilitates melanoma progression by mediating melanoma cell adhesion (Tinari, N., et al., Int J Cancer, 2001, 91, 167-72; van den Brule, F. A., et al., 1995, 209, 760-7; Lotan, R., et al., Glycoconj J, 1994, 11, 462-8; Woynarowska, B., et al., J Biol Chem, 1994, 269, 22797-803) and inducing apoptosis of anti-melanoma T cells (Rubinstein, N., et al., Cancer Cell, 2004, 5, 241-51; Perillo, N. L., et al., Nature, 1995, 378, 736-9; Toscano, M. A., et al., Nat Immunol, 2007, 8, 825-34; Ilarregui, J. M., et al., Nat Immunol, 2009, 10, 981-91; van der Leij, J., et al., Mol Immunol, 2007, 44, 506-13; Juszczynski, P., et al., Proc Natl Acad Sci USA, 2007, 104, 13134-9). However, no skilled artisan has successfully identified Gal-1 ligands expressed on melanoma cell surface until now.

As described herein, melanoma malignancy can be correlated with the expression level of Gal-1 ligands. Because Gal-1 ligands are overexpressed on malignant melanoma cell surface but not on normal melanocytes or melanocytes in compound nevi, this difference in expression levels of Gal-1 ligands is herein exploited to assess or predict the likelihood of a tumor (e.g., skin tumor) to metastasize, in part by monitoring the expression level of Gal-1 ligands of a sample from a subject.

Provided herein is an assay that includes measuring, in a sample obtained from a subject, an expression level of Gal-1 ligands, and determining that the subject has cancer if the expression level of Gal-1 ligands is higher than a reference level.

Also described herein is an assay that includes obtaining a sample comprising a tumor cell from a subject desiring to know the likelihood of cancer, measuring the level of galectin-1 (Gal-1) ligands in the sample and determining that the subject has an increased likelihood of cancer if the expression level of Gal-1 ligands in the sample is higher than a reference level. In an embodiment the assay further includes prescribing a therapy to the subject if the subject has an increased likelihood of cancer. In an embodiment, the assay further comprises administering an effective amount of the therapy to the subject if the subject has an increased likelihood of cancer.

Also described herein is a method of determining whether a subject has cancer. The method includes obtaining a sample comprising a tumor cell from a subject desiring to know the likelihood of cancer, measuring the level of Gal-1 ligands in the sample and determining that the subject has cancer if the expression level of Gal-1 ligands is higher than a reference level. In an embodiment the method further includes prescribing a therapy to the subject if the subject has cancer. In an embodiment, the method may also include administering an effective amount of the therapy to the subject if the subject has an increased likelihood of cancer.

Also provided herein is a method of treating cancer in a subject. The method includes assaying a sample obtained from the subject for an expression level of Gal-1 ligands and comparing the expression level of Gal-1 ligands with a reference level, and when the expression level of Gal-1 ligands is higher than the reference level, administering to the subject an effective amount of a composition comprising an agent that targets the Gal-1 ligands or an agent that targets Gal-1. In some embodiments, the Gal-1 ligands and/or Gal-1 are on a tumor cell.

A related aspect of the invention regards methods that use the assays described herein to determine the malignancy of a tumor. The method comprises assaying a sample obtained from a subject for an expression level of Gal-1 ligands, and determining that the subject has cancer if the expression level of Gal-1 ligands is higher than a reference level.

A method is provided to assess or predict metastatic potential of a tumor from a subject. The method includes measuring, at a first time point, a first expression level of Gal-1 ligands of a first sample from the subject; measuring, at a second time point, a second expression level of Gal-1 ligands of a second sample from the subject, wherein the second time point is later than the first time point; comparing the first expression level with the second expression level, and if the second expression level is higher than the first expression level, the tumor is likely to metastasize. In some embodiments, the first time point can be any time before the tumor has metastasized. The time between the first time point and the second time point can be one day or more, two days or more, a week or more, a month or more, two months or more, three months or more, six months or more, nine months or more or a year or more. It should be noted that the expression level of Gal-1 ligands can be measured at more than two time points. For example, there can be 3, 4, 5, 6, 7, 8, 9, 10, or more time points.

In some embodiments, the average rate of expression level increase of Gal-1 ligands can be quantified. A high average rate indicates a high metastatic potential, while a low average rate indicates a low metastatic potential. Metastatic potential is a measure for tumor progression or metastasis. A high metastatic potential means that the tumor has a high likelihood to metastasize, while a low metastatic potential means that the tumor has a low likelihood to metastasize. In some embodiments, Gal-1 ligand expression would be highest (for example, brightest in the immunofluorescence detection setting) compared with uninvolved adjacent tissue/skin reference in primary cutaneous lesions that apt to or already show signs of metastasis in lesions in extra-cutaneous sites.

Another aspect of the invention provides a composition that comprises a tumor cell and Gal-1hFc. In some embodiments, Gal-1hFc is bound to the tumor cell. In some embodiments, Gal-1hFc is bound to the Gal-1 ligands expressed on the tumor cell surface. In some embodiments, the composition can be made by contacting a tumor cell with Gal-1hFc, and washing the tumor cell to remove excess unbound Gal-1hFc. The tumor cell can be in a cell culture, a tissue sample, or a scaffold.

In various embodiments, Gal-1 ligands include cell surface proteins that display carbohydrate moieties that bind Gal-1. In some embodiments, the carbohydrate moieties include but are not limited to N-acetyllactosamine type 1 (Gal1β1, 3GlcNAc), N-acetyllactosamine type 2 (Gal1β1, 4GlcNAc), or a combination thereof, including but not limited to a linear or branched configuration. In some exemplary embodiments, cells surface proteins that comprise carbohydrate moieties recognized by Gal-1 include but are not limited to CD3, CD4, CD45, melanoma cell adhesion molecule (MCAM), CD43, carcinoembryonic antigen (CEA), LAMP-1, LAMP-2 and glycoprotein 90K/MAC-2BP.

In some embodiments, an increase in Gal-1 ligands is due to an increase in the expression of cell surface proteins that comprise carbohydrate moieties recognized by Gal-1. In some embodiments, an increase in Gal-1 ligands is due to increased carbohydrate moieties on cell surface proteins with carbohydrate moieties recognized by Gal-1. In some embodiments, an increase in Gal-1 ligands is due to the combination of an increase in the expression of cell surface proteins that comprise carbohydrate moieties recognized by Gal-1 and increased carbohydrate moieties on cell surface proteins with carbohydrate moieties recognized by Gal-1.

In various embodiments, the tumor is a skin tumor or an ovarian tumor. In some embodiments, the cancer is an ovarian cancer or a malignant cancer. In some embodiments of melanoma diagnosis, the assays or methods described herein can be used to distinguish melanoma mimics (e.g., Spitz tumors, Seborrheic keratosis, or dermatofibromas) from malignant melanoma.

In some embodiments, the subject is a mammal. In some embodiments, the subject is a human.

### Detection Methods

Any method of assaying a cell for an expression level of a receptor can be used in the present invention. For example, these methods can include, but are not limited to, assaying mRNA expression levels (e.g., fluorescence in situ hybridization), receptor staining (e.g., immunofluorescence), histology, immunohistochemistry, flow cytometry, Western blot, or any combination thereof. Similarly, a skilled artisan can identify other routine methods for identifying expression levels of a receptor or develop methods for Gal-1 ligands expression analysis without undue experimentation. For example, a method of measuring the expression level of the gene responsible for Gal-1 ligands expression can be used in the invention.

In some embodiments, techniques that may be used to assess the amount of nucleic acid encoding, for example, the receptors comprising the Gal-1 ligands, present in the sample include but are not limited to in situ hybridization (e.g., Angerer (1987) Meth. Enzymol 152: 649). Preferred hybridization-based assays include, but are not limited to, traditional “direct probe” methods such as Southern blots or in situ hybridization (e.g., FISH and FISH plus SKY), and “comparative probe” methods such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches. Probes that may be used for nucleic acid analysis are typically labeled, e.g., with radioisotopes or fluorescent reporters. Preferred probes are sufficiently long so as to specifically hybridize with the target nucleic acid(s) under stringent conditions. The preferred size range is from about 200 bases to about 1000 bases. Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In situ Hybridization Protocols, Choo, ed., Humana Press, Totowa, N.J. (1994), Pinkel, et al. (1998) Nature Genetics 20: 207-211, and/or Kallioniemi (1992) Proc. Natl Acad Sci USA 89:5321-5325 (1992).

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the invention. In fluorogenic quantitative PCR, quantitation is based on amount of fluorescence signals, e.g., TaqMan and sybr green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

Suitable methods for assaying the expression level of nucleic acid (for example, nucleic acids encoding the receptors comprising Gal-1 ligands) include but are not limited to using DNA sequencing, comparative genomic hybridization (CGH), array CGH (aCGH), SNP analysis, mRNA expression assay, RT-PCR, real-time PCR, or a combination thereof. In various embodiments, the assay to detect the nucleic acid encoding or protein levels of, GalNAc-T13, is any one or more of Northern blot analysis, Southern blot analysis, reverse transcription-polymerase chain reaction (RT-PCR), polymerase chain reaction (PCR), enzyme-linked immunosorbent assay (ELISA), radio-immuno assay (RIA), sodium dodecyl sulfate polyacrylamide gel electrophoresis (SDS-PAGE), Western blot analysis or a combination thereof.

In some embodiments, the expression level of Gal-1 ligands is measured by immunofluorescence with Gal-1hFc, or a functional fragment or derivative or variant thereof. Gal-1hFc is a fusion protein that links Gal-1 to the Fc region of human IgG1. Gal-1hFc was found to bind to native Gal-1-binding determinants with high fidelity. Gal-1hFc has been used to identify Gal-1-binding determinants and glycoproteins bearing these glycans. For example, see Barthel, S. R., et al, J Biol Chem, 2011, 286, 21717-31; Cedeno-Laurent, F., et al., J Immunol, 2010, 185, 4659-72; Cedeno-Laurent, F., et al., J Invest Dermatol, 2012, 132, 410-420; and Cedeno-Laurent, F., et al., J Immunol, 2012, 188, 3127-37, the content of each of which is incorporated by reference for the teachings of Gal-1hFc and its use thereof. In some embodiments, staining with Gal-1hFc can be done by contacting the sample with Gal-1hFc, and subsequent washing can remove excess unbound Gal-1hFc. Using Gal-1hFc as a model, genetically merging (in frame) any lectin with hFc could create a more stable and functionally potent lectin mimetic. Moreover, the hFc moiety in this chimeric molecule could be probed in numerous bioassays using anti-hFc antibodies.

In some embodiments of using immunofluorescence to measure expression levels, a sample can be stained with Gal-1hFc and one or more other histological probes. For example, histological probes for melanoma cells include, but not limited to, MART-1, MITF, and S100. By way of example only, the inventors have successfully stained melanoma samples with both Gal-1hFc and S100, as described herein.

In some embodiments, the measurement of the expression level of Gal-1 ligands generates a detectable signal. In these embodiments, the detectable signal is produced by a detectable label including, but not limited to, a light-absorbing dye, a fluorescent dye, or a radioactive label. Detectable labels, methods of detecting them, and methods of incorporating them into reagents (e.g. antibodies and nucleic acid probes) are well known in the art. In some embodiments, detectable signals can be detected by spectroscopic, photochemical, biochemical, immunochemical, electromagnetic, radiochemical, or chemical means, such as fluorescence, chemifluoresence, or chemiluminescence, or any other appropriate means.

In some embodiments of using immunofluorescence to measure expression levels, the detectable signal is fluorescence. For example, Gal-1hFc, tagged by a fluorescent dye, and after binding to a cell expressing Gal-1 ligands, can generate fluorescence to indicate the presence of Gal-1 ligands. The immunofluorescence techniques can be primary or secondary.

In some embodiments, the expression level of Gal-1 ligand is assayed by detecting the level of Gal-1 bound to its ligand. In some embodiments, antibodies specific to Gal-1 ligands or to Gal-1 may be used to assay the level of Gal-1 ligands in, for example tumor cells (such as skin tumor cells or ovarian tumor cells). Antibodies, both polyclonal and monoclonal, can be produced by a skilled artisan either by themselves using well known methods or they can be manufactured by service providers who specialize making antibodies based on known protein sequences. In the present invention, the protein sequences of for example, Gal-1, are known and thus production of antibodies against them is a matter of routine.

For example, production of monoclonal antibodies can be performed using the traditional hybridoma method by first immunizing mice with an antigen which may be an isolated protein of choice or fragment thereof (for example, Gal-1 ligand or Gal-1 or a fragment thereof or a variant thereof) and making hybridoma cell lines that each produce a specific monoclonal antibody. The antibodies secreted by the different clones are then assayed for their ability to bind to the antigen using, e.g., ELISA or Antigen Microarray Assay, or immuno-dot blot techniques. The antibodies that are most specific for the detection of the protein of interest can be selected using routine methods and using the antigen used for immunization and other antigens as controls. The antibody that most specifically detects the desired antigen and protein and no other antigens or proteins is selected for the processes, assays and methods described herein.

The best clones can then be grown indefinitely in a suitable cell culture medium. They can also be injected into mice (in the peritoneal cavity, surrounding the gut) where they produce an antibody-rich ascites fluid from which the antibodies can be isolated and purified. The antibodies can be purified using techniques that are well known to one of ordinary skill in the art.

In the methods and assays of the invention, the presence of Gal-1 ligands or Gal-1 (for example, Gal-1 bound to the Gal-1 ligands) is determined using antibodies specific for the Gal-1 ligands or for the Gal-1 protein or a fragment or variant thereof and detecting immunospecific binding of each antibody to its respective cognate marker.

Any suitable immunoassay method may be utilized, including those which are commercially available, to determine the level Gal-1 ligands or Gal-1 (for example, Gal-1 bound to Gal-1 ligands) measured according to the invention. Extensive discussion of the known immunoassay techniques is not required here since these are known to those of skill in the art. Typical suitable immunoassay techniques include sandwich enzyme-linked immunoassays (ELISA), radioimmunoassays (RIA), competitive binding assays, homogeneous assays, heterogeneous assays, etc. Various known immunoassay methods are reviewed, e.g., in Methods in Enzymology, 70, pp. 30-70 and 166-198 (1980).

The antibodies can be labeled. In some embodiments, the detection antibody is labeled by covalently linking to an enzyme, label with a fluorescent compound or metal, label with a chemiluminescent compound. For example, the detection antibody can be labeled with catalase and the conversion uses a colorimetric substrate composition comprises potassium iodide, hydrogen peroxide and sodium thiosulphate; the enzyme can be alcohol dehydrogenase and the conversion uses a colorimetric substrate composition comprises an alcohol, a pH indicator and a pH buffer, wherein the pH indicator is neutral red and the pH buffer is glycine-sodium hydroxide; the enzyme can also be hypoxanthine oxidase and the conversion uses a colorimetric substrate composition comprises xanthine, a tetrazolium salt and 4,5-dihydroxy-1,3-benzene disulphonic acid. In one embodiment, the detection antibody is labeled by covalently linking to an enzyme, label with a fluorescent compound or metal, or label with a chemiluminescent compound.

Direct and indirect labels can be used in immunoassays. A direct label can be defined as an entity, which in its natural state, is visible either to the naked eye or with the aid of an optical filter and/or applied stimulation, e.g., ultraviolet light, to promote fluorescence. Examples of colored labels which can be used include metallic sol particles, gold sol particles, dye sol particles, dyed latex particles or dyes encapsulated in liposomes. Other direct labels include radionuclides and fluorescent or luminescent moieties. Indirect labels such as enzymes can also be used according to the invention. Various enzymes are known for use as labels such as, for example, alkaline phosphatase, horseradish peroxidase, lysozyme, glucose-6-phosphate dehydrogenase, lactate dehydrogenase and urease. For a detailed discussion of enzymes in immunoassays see Engvall, Enzyme Immunoassay ELISA and EMIT, Methods of Enzymology, 70, 419-439 (1980).

The antibody can be attached to a surface. Examples of useful surfaces on which the antibody can be attached for the purposes of detecting the desired antigen include nitrocellulose, PVDF, polystyrene, and nylon. The surface or support may also be a porous support (see, e.g., U.S. Pat. No. 7,939,342). The assays can be carried out in various assay device formats including those described in U.S. Pat. Nos. 4,906,439; 5,051,237 and 5,147,609 to PB Diagnostic Systems, Inc.

In some embodiments of the processes, assays and methods described herein, detecting the level of antibodies reactive to Gal-1 ligands or Gal-1 includes contacting the sample from the cancer patient or patient suspected of having cancer (for example, malignant melanoma or ovarian carcinoma) with an antibody or a fragment thereof that specifically binds Gal-1 ligands or Gal-1, forming an antibody-protein complex between the antibody and Gal-1 ligands or Gal-1 present in the sample, washing the sample to remove the unbound antibody, adding a detection antibody that is labeled and is reactive to the antibody bound to Gal-1 ligands or Gal-1 in the sample, washing to remove the unbound labeled detection antibody and converting the label to a detectable signal, wherein the detectable signal is indicative of the level of Gal-1 ligands or Gal-1 in the sample from the patient. In some embodiments, the effector component is a detectable moiety selected from the group consisting of a fluorescent label, a radioactive compound, an enzyme, a substrate, an epitope tag, electron-dense reagent, biotin, digonigenin, hapten and a combination thereof. In some embodiments, the detection antibody is labeled by covalently linking to an enzyme, labeled with a fluorescent compound or metal, labeled with a chemiluminescent compound. The level of Gal-1 ligands or Gal-1 (for example, Gal-1 bound to Gal-1 ligand) may be obtained by measuring a light scattering intensity resulting from the formation of an antibody-protein complex formed by a reaction of Gal-1 ligands or Gal-1 in the sample with the antibody, wherein the light scattering intensity of at least 10% above a control light scattering intensity indicates the likelihood of chemotherapy resistance.

### Reference Value

In some embodiments, the reference level can be an expression level of Gal-1 ligands in the appropriate tissue of a healthy subject with no signs or symptoms of the tumor of interest (e.g., melanoma or ovarian carcinoma). For example, the appropriate tissue for diagnosing melanoma can be a skin tissue. And the appropriate tissue for diagnosing ovarian tumor can be an ovarian tissue. In some embodiments, the reference level can be an expression level of Gal-1 ligands in a control sample, a pooled sample of control individuals, or a numeric value or range of values based on the same. In some embodiments of melanoma diagnosis, the reference level can be an expression level of Gal-1 ligands of a tissue adjacent to the sin tumor. In some embodiments of melanoma diagnosis, the reference level can be an expression level of Gal-1 ligands of a normal melanocyte. In some embodiments of melanoma diagnosis, the reference level can be an expression level of Gal-1 ligands of a melanocyte in a compound nevus. In certain embodiments, wherein the progression of melanoma in a subject is to be monitored over time, the reference level can also be an expression level of Gal-1 ligands in a subject's sample comprising at least one skin tumor cell which was taken from skin tissue of the subject at an earlier date. It should be noted that the reference level can be different, depending on the particular type of tumors.

In some embodiments, the expression level of Gal-1 ligands can be determined to be higher than the reference level if the expression of Gal-1 ligands is statistically significantly higher than the reference level. In some embodiments, the expression level of Gal-1 ligands can be determined to be higher than the reference level if the expression of Gal-1 ligands is 10% or more than the reference level, e.g., the expression level of Gal-1 ligands is 10% or more of the reference level, the expression level of Gal-1 ligands is 20% or more of the reference level, the expression level of Gal-1 ligands is 30% or more of the reference level, the expression level of Gal-1 ligands is 40% or more of the reference level, the expression level of Gal-1 ligands is 50% or more of the reference level, the expression level of Gal-1 ligands is 100% or more of the reference level, the expression level of Gal-1 ligands is 150% or more of the reference level, the expression level of Gal-1 ligands is 200% or more of the reference level, the expression level of Gal-1 ligands is 250% or more of the reference level, the expression level of Gal-1 ligands is 300% or more of the reference level, the expression level of Gal-1 ligands is 350% or more of the reference level, or the expression level of Gal-1 ligands is 400% or more of the reference level.

### Sample

In some embodiments, the one or more samples for use with the assays and methods described herein are obtained from a subject having a tumor. In some embodiments, the subject has an ovarian tumor. In some embodiments, the subject has a skin tumor. In some embodiments, the sample is obtained by performing a biopsy on a subject. Examples of biopsy techniques include a core needle biopsy, a stereotactic biopsy, incisional biopsy, and a surgical biopsy (e.g. wide local excision or lumpectomy).

As described herein, samples, such as cancer cells, cancerous tissue, plasma and/or blood, could be collected preferably at the time of biopsy for diagnosis of the cancer. This would allow the best chance to design a course of treatment that would best serve the patient. For example, if expression of Gal-1 ligands has increased, the patient may require a more aggressive treatment course compared to another patient with a cancer that does not have increased expression of Gal-1 ligands. It is also possible to obtain cancerous tissue, plasma and/or blood after cancer treatment (e.g., surgery) or during cancer treatment (e.g., radiation. chemotherapy etc.). This would allow for a change in treatment course or decision on the course of treatment with the prospect of recurrence. In various embodiments, the cancer is a malignant melanoma, and ovarian carcinoma.

The steps involved in the current invention comprise obtaining either through surgical biopsy or surgical resection, a sample of the patient's tumor and matching blood sample from the patient. Alternatively, a sample can be obtained through primary patient harvested lung tumor stem cells, primary patient lung tumor derived cell lines, or archived patient samples in the form of FFPE (Formalin fixed, paraffin embedded) samples, or fresh frozen lung tumor samples. This invention also allows for the possibility of retrospectively evaluating the above mentioned parts of this invention (i.e. likelihood of survival, estimated life expectancy and the potential of acquiring this mutation in the future).

Patient's tumor sample is then used to extract Deoxyribonucleic acid (DNA) using the standard protocol designated “QIAamp DNA Mini and Blood Mini kit” or for FFPE samples “QIAamp DNA FFPE Tissue kit” commercially available from Qiagen®. The above and following procedures require informed consent from patients.

In some embodiments, the tumor is a skin tumor and the cancer is malignant melanoma. Ovarian carcinoma cells express Gal-1 ligands. Accordingly, in some embodiments, the tumor is an ovarian tumor and the cancer is ovarian carcinoma.

### Therapies

Another aspect of the invention relates to treating cancer (for example, malignant melanoma or ovarian carcinoma) by targeting, for example, Gal-1 or Gal-1 ligands, by administering an effective amount of a composition that includes an agent that targets Gal-1 or Gal-1 ligands. In some embodiments, the agent that targets Gal-1 or Gal-1 ligands is any one or more of small molecule, antibody, nucleic acid, peptide, aptamer or a combination thereof. In some embodiments, the nucleic acid is an siRNA. In some embodiments, the antibody is a monoclonal antibody or a fragment thereof, a polyclonal antibody or a fragment thereof, chimeric antibody, humanized antibody and single chain antibody. In some embodiments, the agent is a bispecific antibody that targets Gal-1 and Gal-1 ligands.

In some embodiments, the composition comprises a Gal-1-ligand binding moiety that is linked, directly or indirectly, with a compound toxic to the tumor cells. The compound can be any chemotherapeutic drug that is applicable to treating the particular type of cancers. The compound can be an organic molecule, a biological molecule (e.g., a peptide or a nucleic acid), or a combination thereof. For example, FDA approved drugs for melanoma include, but are not limited to, aldesleukin, dabrafenib, dacarbazine, recombinant interferon alfa-2b, ipilimumab, trametinib, peginterferon alfa-2b, and vemurafenib. FDA approved drugs for ovarian carcinoma include, but not limited to, doxorubicin hydrochloride, carboplatin, cyclophosphamide, cisplatin, cyclophosphamide, gemcitabine hydrochloride, topotecan hydrochloride, and paclitaxel. In some embodiments, the compound can be enclosed in a liposome.

In some embodiments, the composition comprising an effective amount of an agent that targets Gal-1 or Gal-1 ligands to treat cancer (for example, melanoma or ovarian carcinoma) is administered with one or more chemotherapeutic agents, such as those set forth herein. Effective amounts of the composition and the chemotherapeutic agent may be administered sequentially or concurrently.

In some embodiments, the administering is systemic. In some embodiments, the administering is local. A variety of means for administering the composition to subjects are known to those of skill in the art. In some aspects of all the embodiments of the invention, the compositions are administered through routes, including ocular, oral, parenteral, intravenous, intramuscular, subcutaneous, transdermal, airway (aerosol), pulmonary, cutaneous, topical, or injection administration.

Additional therapies that may be used with the compositions comprising an effective amount of an agent that targets Gal-1 or Gal-1 ligands to treat cancer (for example, melanoma or ovarian carcinoma) include but are not limited to surgery, radiation, immunotherapy, vaccine or combinations thereof. The additional therapies may be administered sequentially or simultaneously with therapies comprising administering an effective amounts of a compositions comprising an effective amount of an agent that targets Gal-1 or Gal-1 ligands to treat cancer (for example, melanoma or ovarian carcinoma).

In some embodiments, chemotherapeutic agents may be selected from any one or more of cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: treosulfan, and trofosfamide; plant alkaloids: vinblastine, paclitaxel, docetaxol; DNA topoisomerase inhibitors: doxorubicin, epirubicin, etoposide, camptothecin, topotecan, irinotecan, teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.).

As described herein, in various embodiments, therapies include, for example, radiation therapy. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (1-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

As described herein, in various embodiments, therapies include, for example, immunotherapy. Immunotherapy may comprise, for example, use of cancer vaccines and/or sensitized antigen presenting cells. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines.

As described herein, in various embodiments, therapies include, for example, hormonal therapy, Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

The duration and/or dose of treatment with anti-cancer therapies may vary according to the particular anti-cancer agent or combination thereof. An appropriate treatment time for a particular cancer therapeutic agent will be appreciated by the skilled artisan. The invention contemplates the continued assessment of optimal treatment schedules for each cancer therapeutic agent, where the genetic signature of the cancer of the subject as determined by the methods of the invention is a factor in determining optimal treatment doses and schedules.

In various embodiments, the subject for whom predicted efficacy of an anti-cancer therapy is determined, is a mammal (e.g., mouse, rat, primate, non-human mammal, domestic animal such as dog, cat, cow, horse), and is preferably a human. In another embodiment of the methods of the invention, the subject has not undergone chemotherapy or radiation therapy. In alternative embodiments, the subject has undergone chemotherapy or radiation therapy (e.g., such as with cisplatin, carboplatin, and/or taxane). In related embodiments, the subject has not been exposed to levels of radiation or chemotoxic agents above those encountered generally or on average by the subjects of a species. In certain embodiments, the subject has had surgery to remove cancerous or precancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient, or e.g., the subject is given the anti-cancer therapy prior to removal of the cancerous tissue.

### Pharmaceutical Compositions

In various embodiments, the present invention provides pharmaceutical compositions including a pharmaceutically acceptable excipient along with a therapeutically effective amount of an agent that targets Gal-1 or Gal-1 ligands or a combination thereof so as to treat cancer such as malignant melanoma or ovarian carcinoma. In various embodiments, the agent is any one or more of peptides, proteins, antibodies, small molecules, oligonucleotides, nucleic acids or a combination thereof, that recognizes and targets Gal-1 or Gal-1 ligands.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, parenteral or enteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection, either intravenously or intraperitoneally. Methods for these administrations are known to one skilled in the art.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Before administration to patients, formulants may be added to the agents (such as antibodies, nucleic acids or small molecules that target Gal-1 or Gal-1 ligands). A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof. “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

In some embodiments, polymers as formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

Additionally, the agents (such as antibodies, nucleic acids or small molecules that target Gal-1 or Gal-1 ligands) can be chemically modified by covalent conjugation to a polymer to increase their circulating half-life, for example. Preferred polymers, and methods to attach them to peptides, are shown in U.S. Pat. Nos. 4,766,106; 4,179,337; 4,495,285; and 4,609,546 which are all hereby incorporated by reference in their entireties. Preferred polymers are polyoxyethylated polyols and polyethylene glycol (PEG). PEG is soluble in water at room temperature and in some embodiments, has an average molecular weight between 1000 and 40,000, between 2000 and 20,000, or between 3,000 and 12,000. In some embodiments, PEG has at least one hydroxy group, such as a terminal hydroxy group. The hydroxy group may be activated to react with a free amino group on the inhibitor. However, it will be understood that the type and amount of the reactive groups may be varied to achieve a covalently conjugated PEG/antibody of the present invention.

Water soluble polyoxyethylated polyols are also useful in the present invention. They include polyoxyethylated sorbitol, polyoxyethylated glucose, polyoxyethylated glycerol (POG), etc. POG is preferred. One reason is because the glycerol backbone of polyoxyethylated glycerol is the same backbone occurring naturally in, for example, animals and humans in mono-, di-, triglycerides. Therefore, this branching would not necessarily be seen as a foreign agent in the body. The POG has a molecular weight in the same range as PEG. The structure for POG is shown in Knauf et al., 1988, J. Bio. Chem. 263:15064-15070 and a discussion of POG/IL C 2 conjugates is found in U.S. Pat. No. 4,766,106, both of which are hereby incorporated by reference in their entireties.

Another drug delivery system for increasing circulatory half-life is the liposome. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., DRUG DELIVERY SYSTEMS (R. L. Juliano, ed., Oxford, N.Y. 1980), pp. 253-315; M. L. Poznansky, Pharm Revs (1984) 36:277.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

The dosage and mode of administration will depend on the individual. Generally, the compositions are administered so that antibodies are given at a dose between 1 μg/kg and 20 mg/kg, between 20 μg/kg and 10 mg/kg, between 1 mg/kg and 7 mg/kg. In some embodiments, it is given as a bolus dose, to increase circulating levels by 10-20 fold and for 4-6 hours after the bolus dose. Continuous infusion may also be used after the bolus dose. If so, the antibodies may be infused at a dose between 5 μg/kg/minute and 20 μg/kg/minute, or between 7 μg/kg/minute and 15 μg/kg/minute.

### Computer Systems

In some embodiments of the assays and/or methods described herein, the assay/method comprises or consists essentially of a system for determining (e.g., measuring) the expression level of Gal-1 ligands as described herein and comparing them to a reference level or an expression level measured at an earlier time point. If the comparison system, which can be a computer implemented system, indicates that the amount of the measured expression product is higher than that of the reference level, the subject from which the sample is collected can be identified as, e.g. having malignant melanoma. If the comparison system indicates that the amount of the measured expression product is higher than the amount of same expression product measured at an earlier time point, the subject from which the sample is collected can be identified as, e.g. having a melanoma that is likely to metastasize.

In one embodiment, provided herein is a system comprising: (a) at least one memory containing at least one computer program adapted to control the operation of the computer system to implement a method that includes (i) a determination module configured to identify and detect at the expression level of Gal-1 ligands in a sample obtained from a subject; (ii) a storage module configured to store output data from the determination module; (iii) a computing module adapted to identify from the output data whether the expression level of Gal-1 ligands in the sample obtained from the subject is higher, by a statistically significant amount, than a reference level, and (iv) a display module, and (b) at least one processor for executing the computer program.

Embodiments can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules can perform other functions, thus the modules are not limited to having any particular functions or set of functions.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing. Computer-readable storage medium do not include a signal.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the technology discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the technology described herein. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments can include at minimum a determination module, a storage module, a computing module, and a display module. The functional modules can be executed on one, or multiple, computers, or by using one, or multiple, computer networks. The determination module has computer executable instructions to provide e.g., levels of expression products etc in computer readable form.

The determination module can comprise any system for detecting a signal elicited from Gal-1 ligands in a biological sample.

The information determined in the determination system can be read by the storage module. As used herein the “storage module” is intended to include any suitable computing or processing apparatus or other device configured or adapted for storing data or information. Examples of electronic apparatus suitable for use with the technology described herein include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon, for example, sample name, alleleic variants, and frequency of each alleleic variant. Such information may be provided in digital form that can be transmitted and read electronically, e.g., via the Internet, on diskette, via USB (universal serial bus) or via any other suitable mode of communication.

The “computing module” can use a variety of available software programs and formats for computing the expression level of Gal-1 ligands. Such algorithms are well established in the art. A skilled artisan is readily able to determine the appropriate algorithms based on the size and quality of the sample and type of data. The data analysis can be implemented in the computing module. In one embodiment, the computing module further comprises a comparison module, which compares the expression level of Gal-1 ligands in a sample obtained from a subject as described herein with a reference level. In certain embodiments, the reference level can have been pre-stored in the storage module. During the comparison or matching process, the comparison module can determine whether the expression level in the sample obtained from the subject is higher than the reference level to a statistically significant degree. In various embodiments, the comparison module can be configured using existing commercially-available or freely-available software for comparison purpose, and may be optimized for particular data comparisons that are conducted.

The computing and/or comparison module provides a computer readable comparison result that can be processed in computer readable form by predefined criteria, or criteria defined by a user, to provide content based in part on the comparison result that may be stored and output as requested by a user using an output module, e.g., a display module.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to describe the present invention, in connection with percentages means±1%, or ±5%. For example, about 100 means from 95 to 105.

In one respect, the present invention relates to the herein described compositions, methods, and respective component(s) thereof, as essential to the invention, yet open to the inclusion of unspecified elements, essential or not (“comprising”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### EXAMPLES

The following examples are not intended to limit the scope of the claims to the invention, but are rather intended to be exemplary of certain embodiments. Any variations in the exemplified methods which occur to the skilled artisan are intended to fall within the scope of the present invention.

### Example 1

### Gal-1hFc Chimeras as New Tools for Detecting Gal-1 Ligands

The inventors have developed Gal-1-human Fc fusion proteins with high fidelity binding to native Gal-1-binding determinants that can be used to identify Gal-1-binding determinants and glycoproteins bearing these glycans (Barthel, S. R., et al, J Biol Chem, 2011, 286, 21717-31; Cedeno-Laurent, F., et al., J Immunol, 2010, 185, 4659-72; Cedeno-Laurent, F., et al., J Invest Dermatol, 2012, 132, 410-420; Cedeno-Laurent, F., et al., J Immunol, 2012, 188, 3127-37, the contents of each of which are herein incorporated by reference). The amino acid sequence of Gal-1-human Fc fusion is set forth in SEQ ID NO: 1. The nucleic acid sequence of pFUSE-hIgG1-Fc1 plasmid (InvivoGen, San Diego, Calif.) is set forth in SEQ ID NO: 2. The amino acid sequence of hIgG-Fc is set forth in SEQ ID NO: 3. The pFUSE-hIgG1-Fc1 plasmid encodes the Zeocin resistance gene, the amino acid sequence of which is set forth in SEQ ID NO: 4. The cDNA sequence of mouse Galectin-1 (full length) is set forth in SEQ ID NO: 5. The nucleic acid sequence of Galectin-1 fused to human IgG-Fc1 is set forth in SEQ ID NO: 6. The amino acid sequence of mouse Gal-1 is set forth in SEQ ID NO: 7. To control for carbohydrate-binding activity of Gal-1hFc, genetic mutants were created in which a key tryptophan residue for carbohydrate recognition via van der Waals interactions in the 69^{th} amino acid position was substituted for a glycine (mGal-1hFc; SEQ ID NO: 8) and a histidine residue, also important for carbohydrate-binding through the formation of hydrogen bonds in the 45^{th} position was substituted for a leucine (dmGal-1hFc; SEQ ID NO: 9).

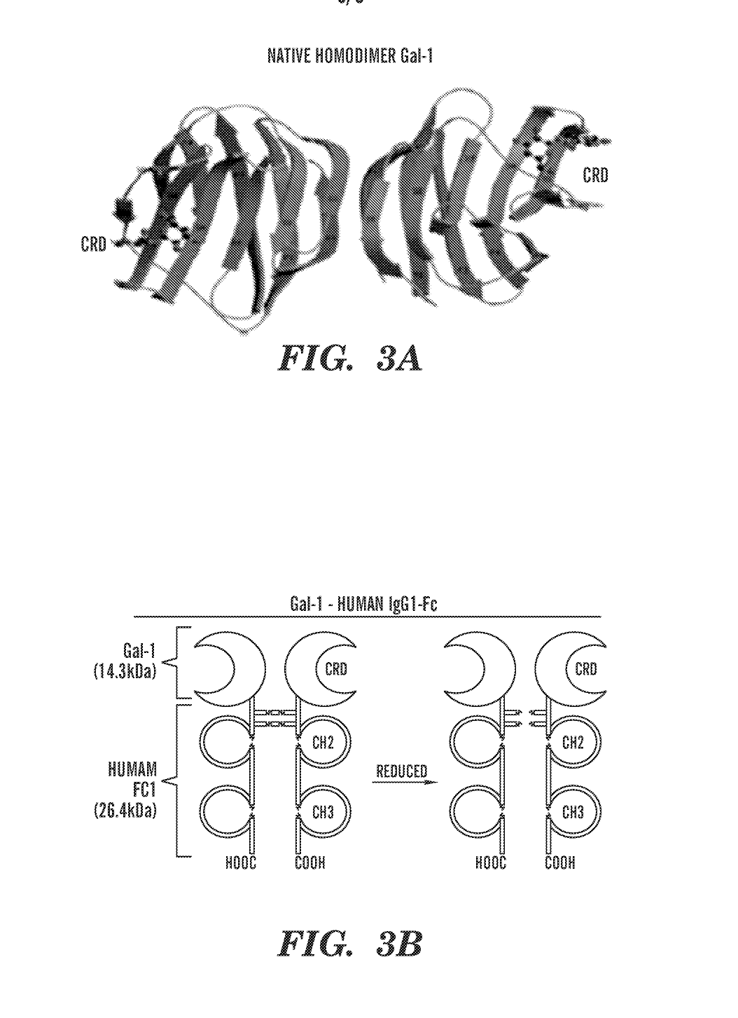

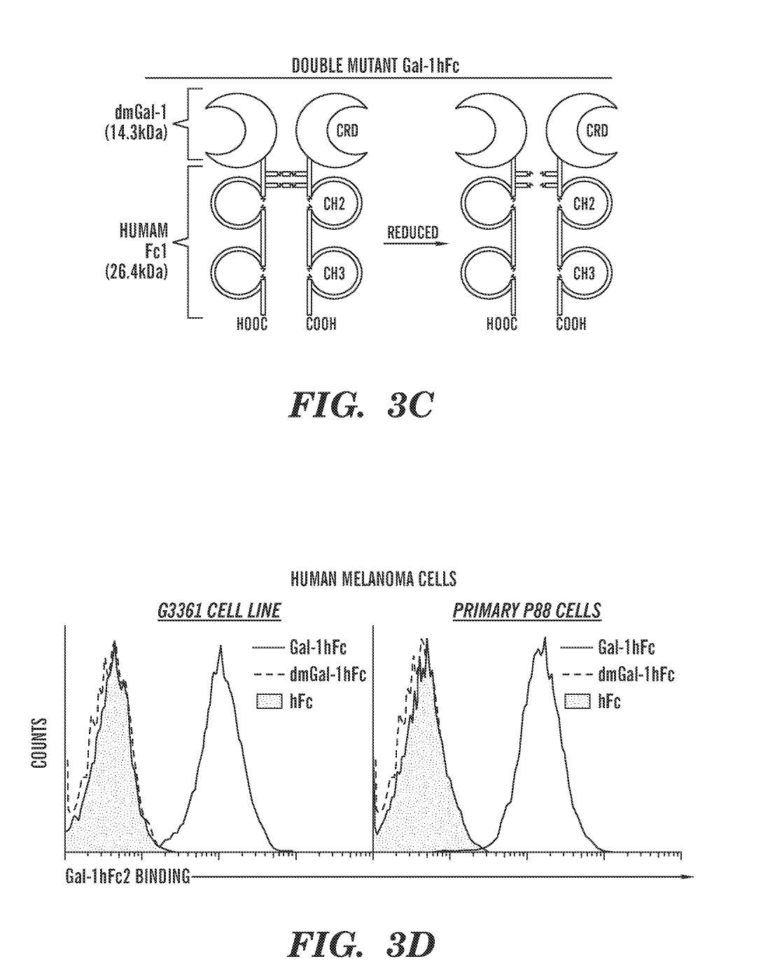

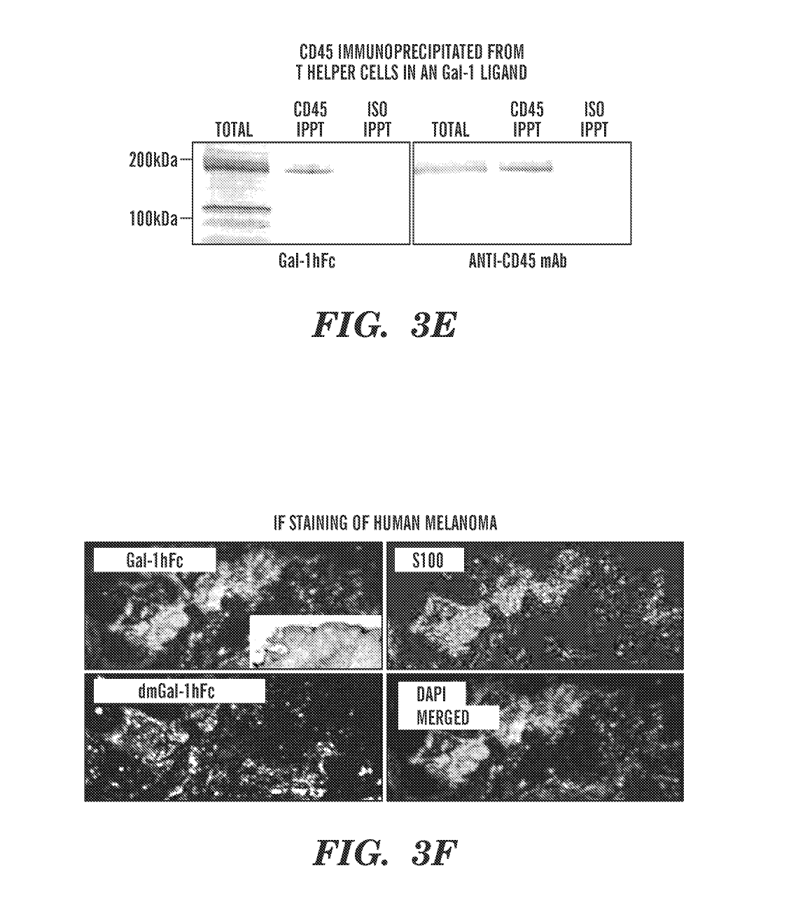

Structural mimetics of native homodimeric Gal-1 were also engineered (FIG. 3A). By linking the Gal-1 to the Fc region of human IgG1, the resultant Gal-1hFc chimera maintains a homodimeric structure that is optimal for Gal-1 ligand binding and induction of Gal-1 ligand-mediated cellular activities (Cedeno-Laurent, F., et al., Blood, 2012, 119, 3534-8) (FIG. 3B). A non-Gal-1 ligand-binding mutant dmGal-1hFc was also created that contains two mutations in key AA residues in the carbohydrate-recognition domain (CRD) to help control for Gal-1-specific binding (FIG. 3C). These chimeras are efficiently secreted from a transfectant, hematopoietic-producer cell line via an IL-2 signal secretion sequence in the Gal-1hFc DNA construct. These Gal-1hFc chimeras are effectively used in bioassays, such as Flow Cytometry (FIG. 3D), Western Blotting (FIG. 3E) and Immunofluorescence (IF) (FIG. 3F) to bind/detect Gal-1 ligands.

### Example 2

### Use of Gal-1hFc as a Histological Probe to Assay for the Presence of Gal-1 Ligands on Melanoma Cells

While Gal-1 ligands have been studied in anti-melanoma T cell immunity, functional expression of Gal-1 ligands on melanoma cells have been largely ignored. In fact, only two prior reports demonstrate adhesion of melanoma cells to Gal-1 and a partial ligand activity by a non-descript 90kda/mac-2 bp molecule (Tinari, N., et al., Int J Cancer, 2001, 91, 167-72; van den Brule, F. A., et al., Biochem Biophys Res Commun, 1995, 209, 760-7). Data from the inventors' studies provide a novel view into expression, identity, regulation and function of Gal-1 ligands on melanoma cells. Furthermore, with the novel Gal-1hfc chimera described in Example 1, a facile immunofluorescent staining approach was developed for analyzing melanocytic tumor malignancy and for predicting metastatic potential based on Gal-1 ligand expression in primary tumors.

### Example 3

### Gal-1 Ligands Natively Expressed on Human Melanoma Cells

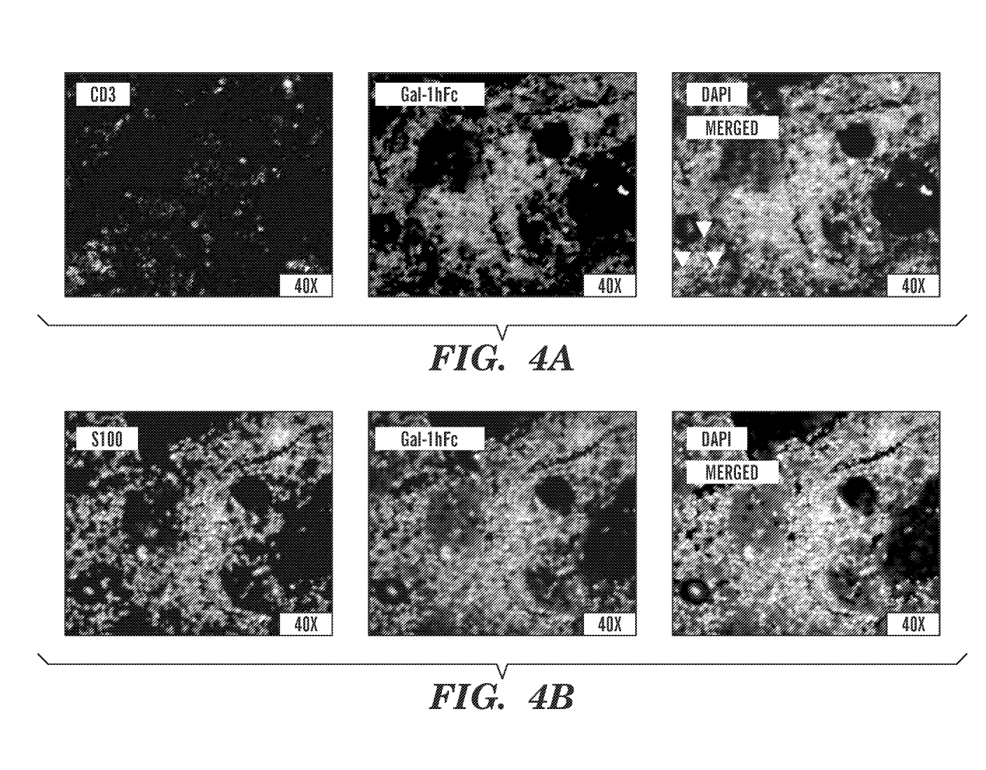

Gal-1hFc was used as a histological probe to assay for the presence of Gal-1 ligand+ T cells in melanoma tissue using (Barthel, S. R., et al., J Biol Chem, 2011, 286, 21717-31; Cedeno-Laurent, F., et al., J Invest Dermatol, 2012, 132, 410-420; Cedeno-Laurent, F., et al., J Immunol, 2012, 188, 3127-37). A FFPE human LN-metastatic melanoma tissue sample was stained with anti-CD3 mAb (FIG. 4A) and Gal-1hFc (FIG. 4A) and found that a subset of human T cells expressed Gal-1 ligand(s) (Arrowheads in FIG. 4A). Surprisingly, the majority of the tumor was largely positive for Gal-1 ligand.

To confirm that Gal-1 ligand was expressed on the melanoma cells, a serial section was double stained with anti-S100A-B Ab (FIG. 4B) and Gal-1hFc (FIG. 4B), which stains normal and malignant melanocytic cells (Cedeno-Laurent, F., et al., J Invest Dermatol, 2012, 132, 410-420), and observed that all S100+ cells stained with Gal-1hFc (FIG. 4B). Staining with negative controls, dmGal-1hFc or Gal-1hFc with 100 mM lactose, did not result in any staining. This is the first demonstration showing that Gal-1 ligands are natively expressed on human melanoma tissue.

### Example 4

### Gal-1 Ligand(s) Detected at a High Level on Malignant Melanocytes and not on Benign Melanocytes

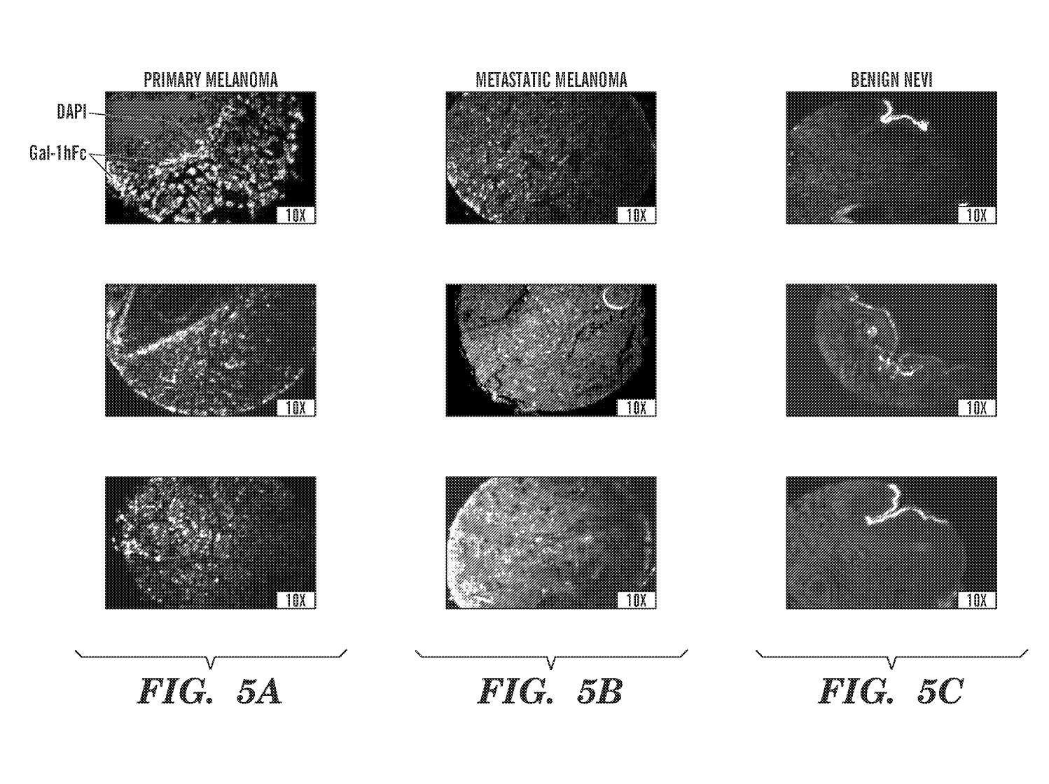

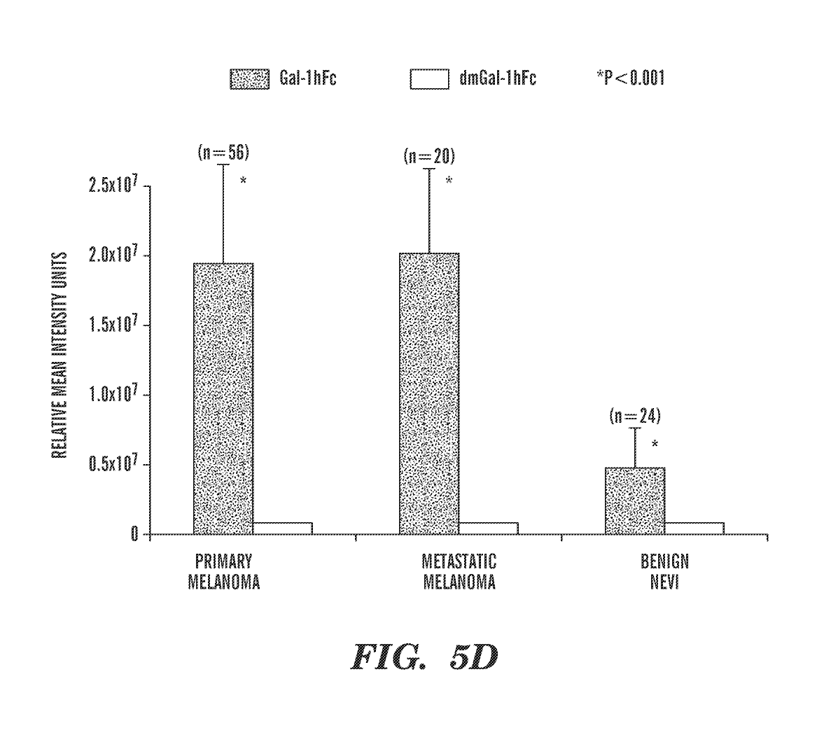

Using tissue microarray (TMA) slides from Biomax, Inc. containing 56 primary melanoma specimens (FIG. 5A), 20 metastatic melanoma samples (FIG. 5B) and 24 benign pigmented lesions (nevi) (FIG. 5C), we stained them with Gal-1hFc or dmGal-1hFc control and APC-goat Fab′ anti-hFc and counterstained with DAPI. Fluorescence analysis was performed using Spot Advanced software, and representative core fields at 10× magnification (encompassing >85% of each core) were analyzed using semi-quantitative raw intensity analysis with NIH Image J software. Relative mean intensities (+/−SD) in the primary and metastatic melanomas were significantly higher than those in the benign lesions (p<0.001) (FIG. 5D).

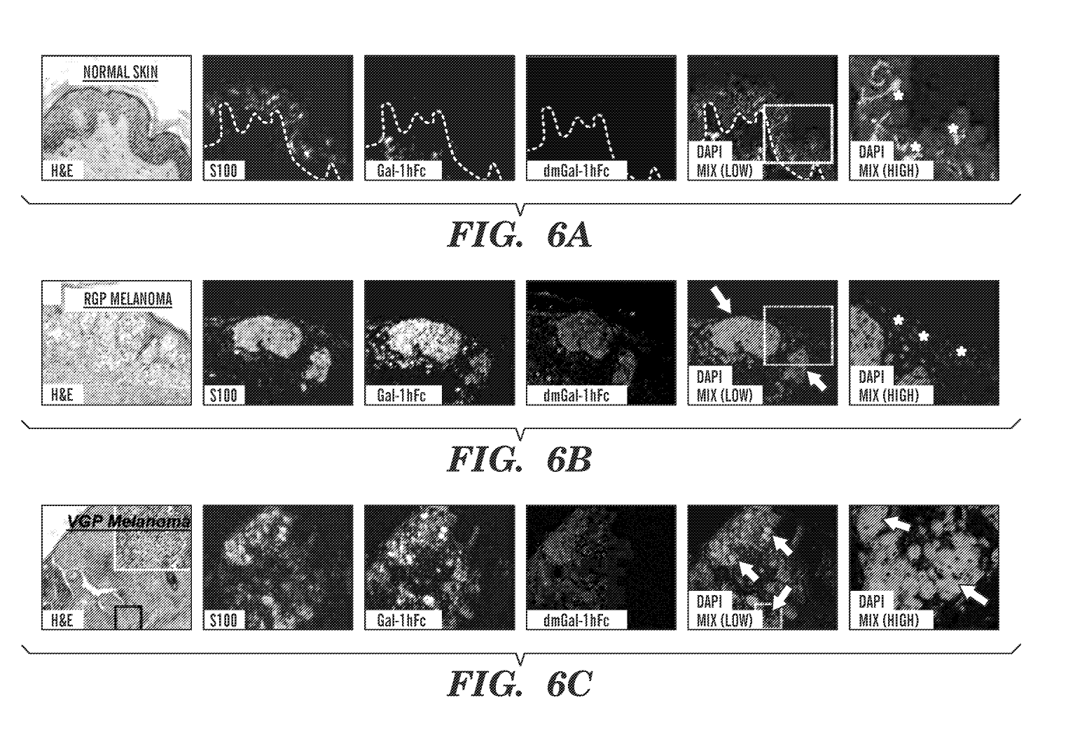

IF analysis of Gal-1 ligand and S100 was performed in human skin and in radial and vertical growth phase (RGP & VGP) melanomas. FFPE tissue sections were double stained with Gal-1hFc or dmGal-1hFc control and anti-S100A-B mAb, which selectively stain Langerhans cells and melanocytic cells. Using normal skin as control, it was found that Gal-1hFc did not stain the epidermis (FIG. 6A), but did show some staining activity in the dermal region, which is likely due to reactivity to effector skin-resident T cells and/or endothelial cells (FIG. 6A at RT) (Cedeno-Laurent, F., et al., J Immunol, 2010 185, 4659-72; Cedeno-Laurent, F., et al., J Invest Dermatol, 2012, 132, 410-420). To the contrary, anti-S100A-B mAb-reactive cells were readily detected in the epidermis of normal skin (FIG. 6A, Asterisks). In both RGP and VGP melanomas, Gal-1hFc stained malignant S100+ melanoma cells (FIGS. 6B& C) but not adjacent S100+ Langerhans cells/melanocytes (FIG. 6B, Asterisks). No staining was observed using dmGal-1hFc (FIGS. 6A-C). In all, this dual IF analysis of (5) primary melanomas and (1) LN-metastatic melanoma showed uniformly that malignant cells were all positive for S100 and Gal-1 ligand (Table 1). Plus, adjacent Gal-1hFc+ and S100+ non-melanoma cells were not evident, showing specificity of this dual staining approach. It was found that Gal-1hFc+ S100− cells are likely T cells and ECs (Cedeno-Laurent, F., et al., J Invest Dermatol, 2012, 132, 410-420; Cedeno-Laurent, F. and C. Dimitroff, Clinical Immunology, 2012, 142, 107-116).

TABLE 1
Dual Gal-1 ligand & S100 staining in human melanomas*

| | Gal-1hFc+ & S100+ | Gal-1hFc+ & S100+ |
|---|---|---|
| | Melanoma cells | Non-malignant cells |
| Diagnosis (n = 6 patients) | (+ out of +++) | (+ out of +++) |
| 1) Invasive melanoma | +++ | − |
| 2) Invasive melanoma | +++ | − |
| 3) Invasive melanoma | +++ | − |
| 4) Melanoma w/in dysplasia | +++ | − |
| 5) Melanoma in situ | +++ | − |
| 6) Metastatic melanoma | +++ | − |

*FFPE primary and metastatic melanomas from (6) de-identified BWH patients were analyzed by dual IF with anti-human S-100A-B (Z0311) (1:3000) and Gal-1hFc. Relative Staining = (−) none, (+) weak; (++) moderate and (+++) strong.

What is claimed is:

1-43. (canceled)

44. A method of determining whether a subject has cancer, comprising:
(i) measuring a level of Gal-1 ligands in a sample comprising a tumor cell obtained from the subject, wherein said measuring is done by contacting the sample with a Gal-1-human Fc (Gal-1hFc) fusion protein; and
(ii) determining that the subject has cancer if the level of Gal-1 ligands is higher than a reference level.

45. The method of claim 44, wherein the Gal-1hFc fusion protein has an amino acid sequence set forth in SEQ ID NO: 1.

46. The method of claim 44, wherein the cancer expresses Gal-1 ligands.

47. The method of claim 44, wherein the tumor is a skin tumor, and wherein the cancer is malignant melanoma.

48. The method of claim 44, wherein the tumor is an ovarian tumor, and wherein the cancer is ovarian carcinoma.

49. The method of claim 44, wherein the sample is obtained by performing a biopsy on the subject.

50. The method of claim 44, wherein the subject is a mammal.

51. The method of claim 50, wherein the mammal is a human.

52. The method of claim 44, wherein the reference level is a level of Gal-1 ligands in samples obtained from a group of control subjects that do not have a tumor.

53. The method of claim 44, wherein the reference level is a level of Gal-1 ligands in samples obtained from a group of control subjects that have the tumor but the tumor is not cancerous.

54. A method of assessing or predicting metastatic potential of a tumor from a subject, comprising:
(i) measuring, at a first time point, a first expression level of Gal-1 ligands of a first sample obtained from the subject, wherein the first expression level is measured by contacting the first sample with a Gal-1-human Fc (Gal-1hFc) fusion protein; and
(ii) measuring, at a second time point, a second expression level of Gal-1 ligands of a second sample obtained from the subject, wherein the f second expression level is measured by contacting the second sample with the Gal-1hFc fusion protein, wherein the second time point is later than the first time point, and wherein when the second expression level is higher than the first expression level, the tumor is likely to metastasize.

55. The method of claim 54, wherein the Gal-1hFc fusion protein has an amino acid sequence set forth in SEQ ID NO: 1.

56. The method of claim 54, wherein the tumor expresses Gal-1 ligands.

57. The method of claim 54, wherein the tumor is melanoma.

58. The method of claim 54, wherein the tumor is ovarian carcinoma.

59. The method of claim 54, wherein the subject is a mammal.

60. The method of claim 59, wherein the mammal is a human.

61. A method of treating cancer in a subject, comprising:
(i) assaying a sample obtained from the subject for an expression level of Gal-1 ligands, wherein the expression level of Gal-1 ligands is measured by: (a) contacting the sample with a Gal-1-human Fc (Gal-1hFc) fusion protein, (b) washing the sample to remove excess unbound Gal-1hFc, and (c) detecting the presence or intensity of a detectable signal; and
(ii) comparing the expression level of Gal-1 ligands with a reference level, and when the expression level of Gal-1 ligands is higher than the reference level, administering to the subject a composition that targets Gal-1 ligands of a tumor cell.

62. The method of claim 61, wherein the Gal-1hFc fusion protein has an amino acid sequence set forth in SEQ ID NO: 1.

63. The method of claim 61, wherein the cancer is malignant melanoma or ovarian carcinoma.


SEQUENCE LISTING (9 sequences)

SEQ ID NO: 1 - protein, 364 aa, Artificial Sequence [sequence omitted]

SEQ ID NO: 2 - DNA, 4132 bp, Artificial Sequence
  Description of Artificial Sequence Synthetic polynucleotide
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggtgaattcgatatctcgagcaccatggttagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatgagtgctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 3 - protein, 227 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 124 aa, Artificial Sequence
  Description of Artificial Sequence Synthetic polypeptide
MAKLTSAVPVLTARDVAGAVEFWTDRLGFSRDFVEDDFAGVVRDDVTLFISAVQDQVVPDNTLAWVWVRGLDELYAEWSEVVSTNFRDASGPAMTEIGEQPWGREFALRDPAGNCVHFVAEEQD

SEQ ID NO: 5 - DNA, 408 bp, Mus sp.
atggcctgtggtctggtcgccagcaacctgaatctcaaacctggggaatgtctcaaagttcggggagaggtggcctcggacgccaagagctttgtgctgaacctgggaaaagacagcaacaacctgtgcctacacttcaatcctcgcttcaatgcccatggagacgccaacaccattgtgtgtaacaccaaggaagatgggacctggggaaccgaacaccgggaacctgccttccccttccagcccgggagcatcacagaggtgtgcatcacctttgaccaggctgacctgaccatcaagctgccagacggacatgaattcaagttccccaaccgcctcaacatggaggccatcaactacatggcggcggatggagacttcaagattaagtgcgtggcctttgagaga

SEQ ID NO: 6 - DNA, 1035 bp, Artificial Sequence
  Description of Artificial Sequence Synthetic polynucleotide
nnnnnnnnnnntgcnnnnnnnnatccagctgtgaccggcgcctacctgagatcaccggtgaattcgatatctcgaggccacccgtctctcgggtggagtcttctgactgctggtggagcaggtctcaggaatctcttcgcttcagcttcaatcatggcctgtggtctggtcgccagcaacctgaatctcaaacctggggaatgtctcaaagttcggggagaggtggcctcggacgccaagagctttgtgctgaacctgggaaaagacagcaacaacctgtgcctacacttcaatcctcgcttcaatgcccatggagacgccaacaccattgtgtgtaacaccaaggaagatgggacctggggaaccgaacaccgggaacctgccttccccttccagcccgggagcatcacagaggtgtgcatcacctttgaccaggctgacctgaccatcaagctgccagacggacatgaattcaagttccccaaccgcctcaacatggaggccatcaactacatggcggcggatggagacttcaagattaagtgcgtggcctttgagagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggangagcagtacaacagcacgtaccgtgtggtcagcgtcctcaccgtcctgcaccaggactggctgaatggcaaggantacaagtgcaaggtctccaacaaagccctcccagcccccatcgagaaaaccatctccaaagccaaagggcagccccganaancacaggtgtacaccctgcccccatcccgggnngagangancnagaaccangtcagccnganntgnntngncaaannctnctatnccnncnnnntc

SEQ ID NO: 7 - protein, 135 aa, Mus sp.
MACGLVASNLNLKPGECLKVRGEVASDAKSFVLNLGKDSNNLCLHFNPRFNAHGDANTIVCNTKEDGTWGTEHREPAFPFQPGSITEVCITFDQADLTIKLPDGHEFKFPNRLNMEAINYMAADGDFKIKCVAFE

SEQ ID NO: 8 - protein, 135 aa, Artificial Sequence
  Description of Artificial Sequence Synthetic polypeptide
MACGLVASNLNLKPGECLKVRGEVASDAKSFVLNLGKDSNNLCLHFNPRFNAHGDANTIVCNTKEDGTGGTEHREPAFPFQPGSITEVCITFDQADLTIKLPDGHEFKFPNRLNMEAINYMAADGDFKIKCVAFE

SEQ ID NO: 9 - protein, 135 aa, Artificial Sequence
  Description of Artificial Sequence Synthetic polypeptide
MACGLVASNLNLKPGECLKVRGEVASDAKSFVLNLGKDSNNLCLLFNPRFNAHGDANTIVCNTKEDGTGGTEHREPAFPFQPGSITEVCITFDQADLTIKLPDGHEFKFPNRLNMEAINYMAADGDFKIKCVAFE